(19) United States
(12) Patent Application Publication
(10) Pub. No.: US 2011/0130357 A1
(43) Pub. Date: Jun. 2, 2011

(54) Aminoglycosides: Synthesis and Use as Antifungals

(75) Inventors: Cheng-wei Tom Chang, Logan, UT (US); C. Kent Evans, Moscow, ID (US); Jon Y. Takemoto, North Logan, UT (US)

(73) Assignee: Utah State University, North Logan, UT (US)

(21) Appl. No.: 12/968,052

(22) Filed: Dec. 14, 2010

Related U.S. Application Data
(63) Continuation-in-part of application No. PCT/US09/46827, filed on Jun. 10, 2009.
(60) Provisional application No. 61/060,661, filed on Jun. 11, 2008.

(51) Int. Cl. A61K 31/7036; C07H 15/234; A01N 43/16; A01P 3/00; A61P 31/00

(52) U.S. Cl. 514/41; 536/13.7

(57) ABSTRACT

The present invention relates to novel aminoglycoside analogs having certain substituents at the 6 position of ring III which exhibit improved antifungal activity but possess minimal antibacterial properties. The compounds of the present invention are analogues of kanamycin B and kanamycin A. Also provided are methods of synthesizing and methods of using the compounds of the present invention. The compounds of the present invention are useful in treating or preventing fungal disease.

### CROSS-REFERENCE TO RELATED APPLICATIONS

This application in a continuation-in-part of International Patent Application PCT/US2009/046827 filed Jun. 10, 2009 which in turn claims the benefit of U.S. Provisional Applications 61/060,661, filed on Jun. 11, 2008, both of which are hereby incorporated herein by reference.

### STATEMENT REGARDING FEDERALLY SPONSORED RESEARCH OR DEVELOPMENT

This invention was made with federal support (NIH Grant R01 AI053138) and the federal government may have certain rights in the invention.

### REFERENCE TO SEQUENCE LISTING, A TABLE, OR A COMPUTER PROGRAM LISTING COMPACT DISK APPENDIX

Not Applicable

### TECHNICAL FIELD

The present invention is in the technical field of antimicrobials. More particularly, the present invention is in the technical field of aminoglycoside antimicrobials and antifungals in particular.

### BACKGROUND ART

Aminoglycoside antibiotics have been commonly used as a medical treatment against infectious diseases for over 60 years, although the prevalence of aminoglycoside resistant bacteria has significantly reduced their effectiveness. Aminoglycosides have two or more amino sugars bound to an aminocyclitol ring through glycosidic bonds. Naturally occurring aminoglycosides (produced by Actinomycetes) are widely used as antibiotics against bacterial infections of animals and humans. These include the well-known antibiotics kanamycin, streptomycin and neomycin. Aminoglycoside antibiotics are believed to act on the bacterial protein synthesis machinery, leading to the formation of defective cell proteins.

In medicine, fungal diseases have emerged over the last 25 years as a major public health problem. Among the prominent reasons for this increase are the lack of efficacious antifungal agents, increases in immunocompromised conditions (e.g., organ transplants and HIV/AIDS), and widespread resistance to the most commonly used antifungals. The strongest medically used antifungal agent, amphotericin B, is an effective medication, but is also highly toxic to patients. The toxicity levels of the available antifungal medications are a common concern for medical practitioners. U.S. Pat. No. 5,039,666 to Novick, Jr. (1991) shows an aminoglycoside compound “gentamicin” having reduced nephrotoxicity induced by the aminoglycoside. Other common antifungal medications are used to treat infections such as athlete's foot, ringworm, candidiasis (thrush) and serious systemic infections such as cryptococcal meningitis, and others.

In agriculture, the control of crop diseases by direct application of biocides remains the most effective and most widely used strategy. Nevertheless, concerns with inconsistent and declining effectiveness, environmental impacts, animal/human toxicity, and costs continue to challenge the use of existing biocides. Traditionally, aminoglycosides have been developed and used as antibiotics against bacteria. A recent report, however, suggests inhibition of plant pathogenic fungi (particularly by paramomycin) by traditional and natural aminoglycosides. One specific example of a crop pathogen is Fusarium graminearum, the most common causative agent of head blight disease in wheat and barley in North America. Infection with F. graminearum is difficult to predict and can result in catastrophic crop loss.

Kanamycin is a known aminoglycoside antibiotic. The antibiotic function of kanamycin is believed to be related to its ability to affect the 30S ribosomal subunit of bacteria, causing frameshift mutations or preventing the translation of RNA. Either frameshift mutations or a lack of RNA translation can lead to a reduction or absence of bacterial protein synthesis and, ultimately, to bacterial death. Unfortunately, kanamycin has been rendered all but obsolete for clinical use due to the emergence of resistant bacteria.

Clearly there exists a need for novel antimicrobials to address the problems of resistant bacteria and fungi, both in human medicine and in crop disease. There is also a clear need for novel antimicrobials, especially antifungals, with reduced toxicity. Furthermore, it would be desirable for new antimicrobial compounds to be selective against either bacteria or fungi, so treatment for one of either bacterial or fungal disease does not contribute to the buildup of antimicrobial resistance in the other. Selective antimicrobial activity is especially desirable for antifungals used to treat crop disease, such as Fusarium head blight, due to the possibly large amounts of antimicrobial agent released into the environment when crops are treated. The present invention provides for novel aminoglycoside antimicrobials that are effective, have relatively low levels of toxicity, and are selective against fungal pathogens.

### BRIEF SUMMARY OF THE INVENTION

The present invention relates to novel aminoglycoside analogs derived from the parent molecules kanamycin B and, to a lesser extent, kanamycin A. It is an object of the present invention to provide novel aminoglycoside analog compounds with Chemical Formula I as follows:

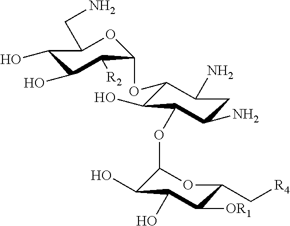

wherein R₄ is a member selected from the group consisting of H and OH; R₂ is a member selected from the group consisting of OH and NH₂; and R₁ is a member selected from the group consisting of R₃ (alkyl), R₃O(CO), (alkoxycarbonyl), R₃NH(CO), (alkylaminocarbonyl), R₃S(O)₂, (alkylsulfonyl), R₃S(O), (alkylsulfinyl), R₃P(O)₂, (alkylphosphonyl), R₃C(O) (alkanoyl), phenyl and C₁ to C₆ alkyl substituted phenyl; wherein R₃ is a straight or branched chain C₄ to C₁₂ alkyl group.

It is also an object of the present invention to provide novel aminoglycoside analog compounds of Formula I having improved antimicrobial and particularly antifungicidal properties.

A still further object of the present invention is to provide method of synthesizing aminoglycoside analog compounds of Formula I. Yet another object of the present invention is to provide methods of using aminoglycoside analog compounds of Formula I as biocides having improved fungicidal activity.

Without limiting the invention to a any particular method of use, the compounds of the present invention unexpectedly demonstrate increased specifity for fungal pathogens and a lack of activity against some common bacterial pathogens. Other synthetic aminoglycosides and the natural aminoglycosides are either solely bacteriocidal or both bacteriocidal and fungicidal. As a result of its unexpected specificity for fungal pathogens, the compounds of the present invention provides for advantageous treatment of fungal pathogens by not promoting resistance of pathogenic bacteria to traditional aminoglycosides and by not harming or eliminating nonpathogenic bacteria. Various embodiments of the present invention, as well as examples for a method of synthesizing and methods of using the compound of the present invention, are discussed below.

### DEFINITIONS

Before discussing the present invention in further details, the following terms and conventions will first be defined:

Host: The term “host” is defined herein as any living organism infected or at least somewhat likely of being infected by a fungal pathogen, where said pathogen and any infection caused by said pathogen, or potential infection caused by said pathogen, are susceptible to treatment with one or more of the compounds of Formula I as claimed herein, where said treatment is likely to result in the elimination, avoidance, or alleviation of the infection caused by said pathogen.
When R₁ is n-octyl (C₈H₁₇), R₂ is NH₂ and R₄ is H, “JL038” or “FG08” are terms used to identify this compound of the present invention and is synonymous with 6-O-(6-Deoxy-4-O-n-octyl-α-D-glucopyranosyl)neamine. “JL038” is a term used to identify the compound of the present invention in research reports and publications. In figures contained within research reports, publications, and the present patent application, “JL038” is sometimes abbreviated as “JL38” or “38,” and is sometimes associated with other numbers that describe concentrations used or other physical parameters. To that end, “JL038” is also synonymous with “FG08.” “M038,” “Th38,” “38”, “FG08,” and 6-O-(6-Deoxy-4-O-n-octyl-α-D-glucopyranosyl)neamine are one and the same compound and are used interchangeably.
More specifically, “FG08” is a name or symbol more recently and preferably used by the inventors to identify this n-octyl analog compound of the present invention.
When R₁ is n-butyl (C₄H₉). R₂ is NH₂ and R₄ is H “FG01” is a term used to identify this compound and is synonymous with 6-O-(6-Deoxy-4-O-n-butyl-α-D-glucopyranosyl)neamine.
When R₁ is n-dodecyl (C₁₂H₂₃), R₂ is NH₂ and F₄ is H, “FG02” is a term used to identify this compound and is synonymous with 6-O-(6-Deoxy-4-O-n-dodecyl-α-D-glucopyranosyl)neamine.
When R₁ is n-octyl (C₈H₁₇), R₂ is NH₂ and F₄ is OH, “FG03” is a term used to identify this compound and is synonymous with 4-O-(6-Amino-6-deoxy-α-D-glucopyranosyl)-6-O-(4-O-n-octyl-α-D-glucopyranosyl)-2-deoxystreptamine.
For comparative purposes, isomeric aminoglycosides, outside the scope of this invention, are identified as JL039 and JL040 and have the formulas;

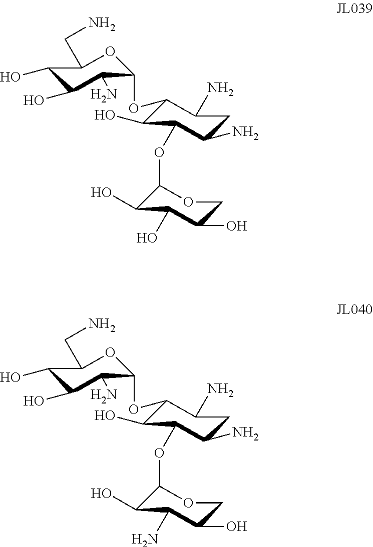

Also, for comparative purposes Kanamycin B, and Kanamycin A have the structures:

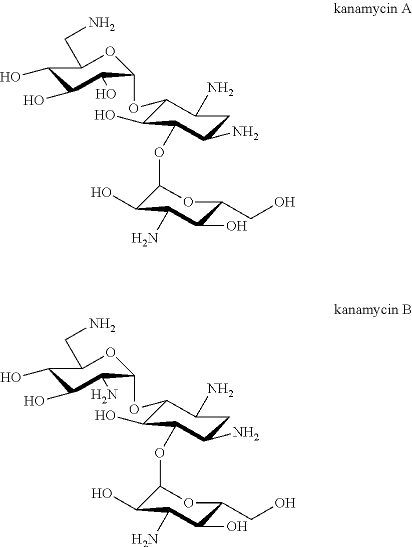

Some or all of the following definitions may also be utilized throughout this disclosure.
Fungal Infection: The term “fungal infection” is defined herein as an association of a fungal organism with a host, whether said association is actual or potential. For example, an actual associate occurs when a fungi is physically present on or within a host. Examples of potential associations include fungi on or within the environment surrounding a host, where the fungi is at least somewhat likely to be actively or passively transferred to the host. Without wishing to further limit the type of associations between a fungal organism and host, examples of the association of the fungal organism with the host include biological associations that may be pathogenic or non-pathogenic, parasitic or non-parasitic, symbiotic or non-symbiotic, mutualistic or non-mutualistic, commensal or non-commensal, naturally occurring or man-made, or any other biological interaction.
Host in need thereof: The phrase “host in need thereof” is defined herein as any host associated or potentially associated with a fungal organism, where said host may actually or potentially benefit from elimination, prevention, or alleviation of a fungal infection.
Fusarium Head Blight: The phrase “fusarium head blight” is defined herein as any fungal disease caused by the fungus Fusarium graminearum.
Surfactant: The term “surfactant” is used to indicate the common laboratory surfactant C₅₈H₁₁₄O₂₆. All uses of the term “surfactant” refer to C₅₈H₁₁₄O₂₆, unless otherwise indicated.
Prophylactically: The term “prophylactically” is used herein to refer to the administration of an antimicrobial compound for the prevention of disease.
N/A: As used herein to describe data points, the abbreviation “N/A” means not tested.
Adjuvant: The term “adjuvant” is defined herein as a substance that helps and enhances the pharmacological effect of a drug or increases the ability of an antigen to stimulate the immune system.
Excipient: The term “excipient” is defined herein as an inactive substance used as a carrier for the active ingredients of a medication.
Diluent: The term “diluent” is defined herein as any liquid or solid material used to dilute or carry an active ingredient.
Antifungal Amount or Antifungal Effective: Unless otherwise specified, the phrases “antifungal amount” or “antifungal effective” are used herein to describe an amount of an antifungal agent sufficient to reduce, eliminate, or alleviate a fungal infection or the symptoms of a fungal infection on or within a host.
MIC: The term MIC means the minimal inhibitory concentration or lowest concentration of an antimicrobial that will inhibit the visible growth of a microorganism after 24, 48, or 72 hours of incubation.
Admixed: The term “admixed” is used herein to describe a chemical or compound in a mixture or combination with other chemicals or compounds.
Administering: The term “administering” is defined herein to describe the act of providing, exposing, treating, or in any way physically supplying or applying a chemical or compound to any living organism or inanimate object associated with a living organism, where said organism will actually or potentially benefit for exposure, treatment, supplying or applying of said chemical or compound.
Topical: The term “topical” is defined herein as pertaining to the surface of a body part, surface part of a plant, or surface of an inanimate object or composition, such as soil. For example, in medicine, a topical medication is applied to body surfaces such as the skin or mucous membranes, for example throat, eyes and ears.
Carrier: The term “carrier” is defined herein as any substance that serves to improve the delivery and the effectiveness of a drug or antimicrobial agent and is inclusive of excipients as defined above.
Examples include:
Microspheres made of the biodegradable polymer poly(lactic-co-glycolic) acid
albumin microspheres;
synthetic polymers (soluble);
Protein-DNA complexes;
protein conjugates;
erythrocytes;
Nanoparticles; and
Liposomes
   Grain head: The phrase “grain head” as used herein is meant to include both small and large grains, e.g. wheat and corn.
   Warm-blooded animal: Used herein the phrase “warm-blooded animal” means an animal characterized by the maintaining of a relatively constant and warm body temperature independent of environmental temperature; homeothermic.

Certain terms in this application are meant to be interpreted as commonly used in the technical fields of medicine, antimicrobials, and crop disease, as indicated by the context of their use. These terms include spray nozzle, droplet, therapeutically, exterior, spraying, topical, treatment, and prevention.

### BRIEF DESCRIPTION OF THE DRAWINGS

FIG. 1 shows a generic chemical structure of the present invention.

FIG. 2 outlines a method by which to synthesize various embodiments of the present invention including FG01, FG02, FG03 and FG08.

FIG. 3 shows the percent lesion areas developed in leaf infection assays with treatments utilizing a species of the present invention (FG08 or 38), other comparative aminoglycosides JL39 or 39, JL40 or 40 and kanamycin B or KB.

FIG. 4 shows the relative chlorophyll contents in leaf infection assays with treatments utilizing a species of the present invention (FG08 or 38)) and other comparative aminoglycosides, JL39 or 39, JL40 or 40 and kanamycin B or KB.

Table 1 shows the estimated MICs of 5 aminoglycosides against F. graminearum; JL038 (FG08), of the present invention and comparative aminoglycosides JL39, JL40, kanamycin A, and kanamycin B.
Table 2 shows a list of screened fungi and the minimal inhibitory concentration (μg/ml) for effective treatment with the compound FG08(J138) of the present invention and comparison with kanamycin B.
Table 3 shows in vitro antifungal activities of FG08 and analogs thereof, FG01, FG02, FG03 and FG04 and Kanamycin B against Fusarium graminearum in terms of minimal inhibitory concentration or MIC(μg/ml).

### DETAILED DESCRIPTION OF THE INVENTION

The present invention relates to a antimicrobial compounds comprising formula I as follows:

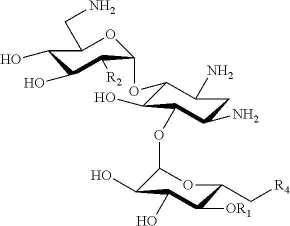

wherein R₄ is a member selected from the group consisting of H and OH; R₂ is a member selected from the group consisting of OH and NH₂ and R₁ is a member selected from the group consisting of R₃ (alkyl), R₃O(CO), (alkoxycarbonyl), R₃NH(CO), (alkylaminocarbonyl), R₃S(O)₂, (alkylsulfonyl), R₃S(O), (alkylsulfinyl), R₃P(O)₂, (alkylphosphonyl), R₃C(O) (alkanoyl), phenyl and C₁ to C₆ alkyl substituted phenyl; wherein R₃ is a straight or branched chain C₄ to C₁₂ alkyl group.

These compounds are substituted analogs of kanamycin A and B. The present invention also relates to methods for the synthesis of such analogs and the utilization of them as antifungal agents.

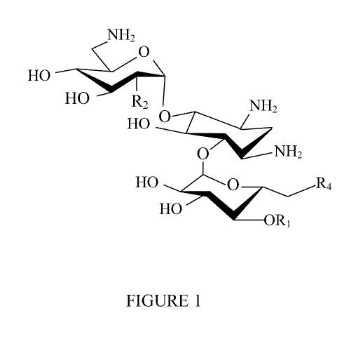

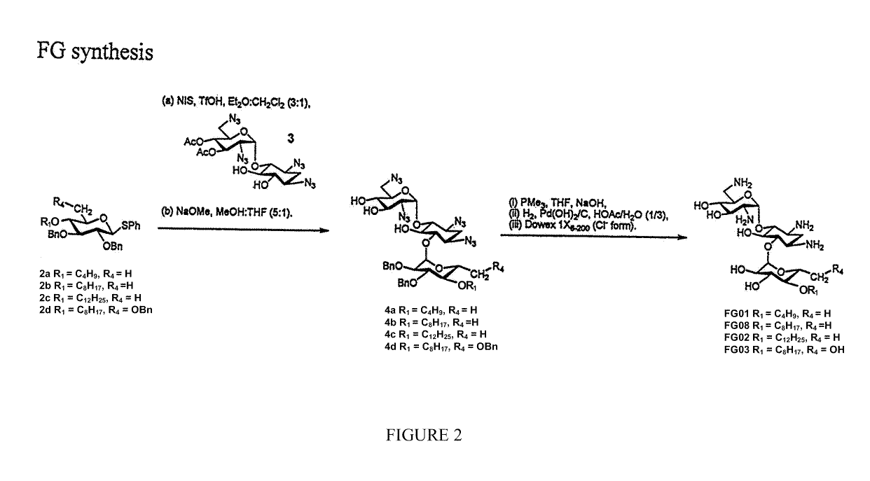

Referring now to the invention in more detail, in FIG. 1 there is shown the structure of the compounds related to the present invention. The compounds related to the present invention are analogs of either the parent molecule kanamycin B (R₂ is NH₂) or kanamycin A (R₂ is OH). The structure related to the present invention is distinguished from the parent molecules kanamycin B or kanamycin A by the presence of functional groups terminating in either an alkyl or phenyl group in the 6 position of ring III. Further, in the 5 position of ring III R₄ is either H or OH such that the substituent in the 5 position is either a methyl or hydroxy methyl group.

More specifically, in reference to FIG. 1, the functional groups at the 6 position of ring III are identified as R₁ and are members selected from the group consisting of R₃ (alkyl), R₃O(CO), (alkoxycarbonyl), R₃NH(CO), (alkylaminocarbonyl), R₃S(O)₂, (alkylsulfonyl), R₃S(O), (alkylsulfinyl), R₃P(O)₂, (alkylphosphonyl), R₃C(O) (alkanoyl), phenyl and C₁ to C₆ alkyl substituted phenyl; wherein R₃ is a straight or branched chain C₄ to C₁₂ alkyl group.

In more preferred embodiments there are no intervening oxycarbonyl O(CO); aminocarbonyl (NH(CO); sulfonyl (S(O)₂; sulfinyl (S(O); phosphonyl (P(O)₂ or carbonyl (C(O) linkages between the O appendage at the 6 position of ring III nor is there a phenyl or alkyl substituted phenyl linked to the O appendage at the 6 position of ring III such that R₁ and R₃ are both straight or branched chain C₄ to C₁₂ alkyl are directly linked to the 0 appendage at the 6 position of ring III. Therefore the preferred aminoglycoside, referring to Formula I wherein R₁ is alkyl and R₁ and R₃ are the same.

In a kanamycin B embodiment R₂ is NH₂ and R₄ is H or OH. In this embodiment R₃ is preferably C₄(n-butyl); C₈(n-octyl) or C₁₂(n-dodecyl). R₄ can be either H or OH but is most preferably H.

In a kanamycin A embodiment R₂ is OH and R₄ is H or OH. In this embodiment R₃ is preferably C₄(n-butyl); C₈(n-octyl) or C₁₂(n-dodecyl). R₄ can be either H or OH but is most preferably OH.

In a most preferred embodiment, R₂ is NH₂ and R₄ is H and R₃ is C₈(n-octyl). This species is identified as 6-O-(6-Deoxy-4-O-n-octyl-α-D-glucopyranosyl)neamine (FG08).

### EXAMPLES

The following materials and methods were used in either one or more of the examples listed below. Further materials and methods are provided in the description of each example.

### Aminoglycosides

All aminoglycosides were kindly provided by the laboratory of Dr. Tom Chang (Department of Chemistry and Biochemistry, Utah State University). They were stored as 10 mg/mL solutions in water at 4° C.

### Growth Medium

Fresh potato dextrose broth (PDB)+casamino acids was used throughout. To make 11 of PDB+casamino acids, 200 g of fresh diced potatoes were boiled in 500 ml of distilled water for 30 min. The broth was filtered through 2 layers of cheese cloth, and the volume was brought up to 11. After additions of 20 g of glucose (2%, w/v) and 4 g of casamino acid (0.4%, w/v), the mixture was stirred with a magnetic bar until all solids were dissolved. Then, the medium was sterilized by autoclaving for 30 min. Potato dextrose agar (PDA)+casamino acids medium was prepared with 1.5% agar and poured into plastic petri plates.

Isolates of F. graminearum

Two isolates of F. graminearum, B-4-5A and Butte86ADA-11 were obtained from the Small Grain Pathology Program of the University of Minnesota. Both strains were used for MIC tests and only B-4-5A was used for leaf infection assays

### Example 1

### Synthesis of 6-O-(6-Deoxy-4-O-n-octyl-α-D-glucopyranosyl)neamine (FG08 or JL038)

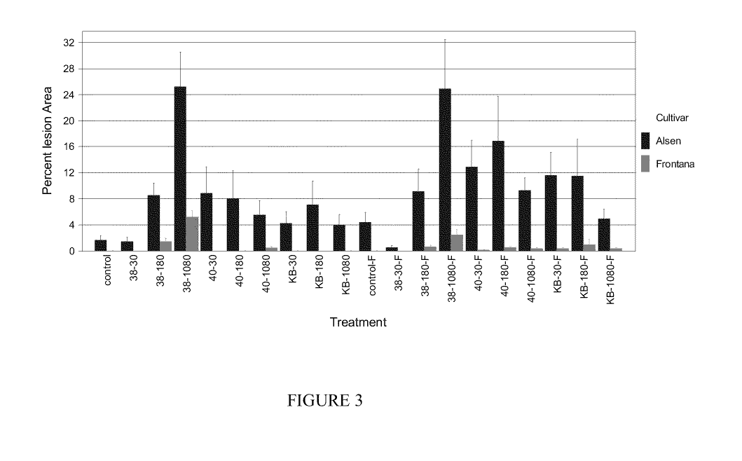

Referring now to FIG. 2, the compounds of the invention can be synthesized by glycosylation of 3 using an appropriately substituted 2 (shown as 2a-d) as the glycosyl donor. Following the deprotection of acetyl group, compound 4 (shown as 4a-d) can be subjected to Staudinger reduction and hydrogenation. The crude product can be purified using CG-50 resin (eluted with gradient of aqueous NH₄OH solution). The final product in chloride form can be obtained by passing through Dowex 1X-8 (Cl⁻) to obtain FG01, FG02, FG03 and FG08). Other members or species can be synthesized similarly using the corresponding substituted glycosyl donors and appropriate kanamycin A or B structure. It is to be noted that while FIG. 2 outlines a flowchart for the synthesis of the compounds of this invention, not every step or chemical utilized in the synthesis and recovery of the final product is contained in the flowchart. The flowchart is sufficient to enable one skilled in the art to synthesize the compounds of the invention. However, a more detailed procedure for the synthesis of 6-O-(6-Deoxy-4-O-n-octyl-α-D-glucopyranosyl)neamine (FG08) is given below. One skilled in the art can readily adapt and use this detailed synthesis as a means for the preparation of other analogs disclosed and claimed herein.

Referring again to FIG. 2, the general procedure for glycosylation and hydrolysis is as follows: A solution of glycosyl donor, neamine derivative (1.2 equivalents), and activated powder 4A° molecular sieve was stirred in anhydrous Et₂O and CH₂Cl₂ (Et₂O: 4.5 ml; CH₂Cl₂: 1.5 ml) at room temperature overnight. N-iodosuccinimide (1.2 equivalents) was quickly added into the above solution and the reaction mixture was cooled to −70° C. After the temperature of the solution warmed up to −40° C., trifluoromethanesulfonic acid (0.15 equivalents) was added. The solution was stirred at low temperature till the complete consumption of the glycosyl donor (ca. 4 hours, monitored by TLC, Hexane: EtOAc=65:35). The reaction mixture was quenched by the addition of triethyl amine (3 mL). After being stirred for 10 minutes, the reaction mixture was filtered through Celite and the solvent was removed. The crude product was extracted with EtOAc, washed with 10% aqueous Na₂S₂O₃, saturated NaHCO_{3(aq)} and brine, and dried over Na₂SO₄₍ₛ₎. After removal of the solvents, the crude product was purified with column chromatography. The glycosylated compounds were often mixed with inseparable impurities, and were fully characterized after hydrolysis. The glycosylated product was dissolved in tetrahydrofuran (1 ml) and methanol (5 ml), and sodium methoxide (0.5 M in methanol, 1 ml) was added. The reaction mixture was stirred at room temperature till the completion of the reaction (ca. 2 hours, monitored by TLC, EtOAc:Hexane=50:50). The reaction mixture was neutralized with Amberlite IR-120 (H⁺), filtered through Celite and the solvent was removed. The residue was purified via column chromatography to provide the product as colorless oil.

Still referring to FIG. 2, the general procedure for the final synthesis: A solution of starting material and K₂CO₃ (4-5 equivalence) was stirred in MeOH (5 ml) at room temperature till the complete consumption of starting material (ca. 5 hours). The solvent was removed, and the reaction mixture was diluted with EtOAc and filtered through a short column packed with TLC silica gel and Celite. The column was eluted with EtOAc or EtOAc/MeOH (1/1 solution) depending on the polarity of the product. Removal of solvents afforded crude product mixed with some K₂CO₃ solid. The crude product was added with THF (6 ml) and filtered through glasswool into the reaction flask for the next step. To the perbenzylated azidoaminoglycoside/THF solution in a reaction vial equipped with a reflux condenser, 0.1 M NaOH_{(aq)} (0.5 ml) and PMe₃ (1M in THF, 5-6 equivalent) were added. The reaction mixture was stirred at 50° C. for 2 hrs. The product has an Rf value of 0 when eluted with EtOAc/MeOH (9/1) solution and an Rf value of 0.9 when eluted with iPrOH/1M NH₄OAc (2/1) solution. After completion of the reaction, the reaction mixture was cooled to room temperature and loaded to a short column (5 cm in height) packed silica gel and Celite. The column was eluted with a series of solutions as the follows: THF, THF/MeOH, MeOH, and MeOH/conc.NH₄OH (from 0 to 28% of conc. NH₄OH). The fractions containing desired product were analyzed by TLC and collected. After removal of solvents, the crude perbenzylated aminoglycoside was added with catalytic amount of Pd(OH)₂/C (20% Degussa type) and 5 ml of degassed HOAc/H₂O (1/1). After being further degassed, the reaction mixture was stirred at room temperature under atmospheric H₂ pressure. After being stirred for 1 day, the reaction mixture was filtered through Celite. The residue was washed with water, and the combined solutions were concentrated affording pure final product as an acetate salt. The product has an Rf value of 0 when eluted with iPrOH/1M NH₄OAc (2/1) solution and a Rf of 0.1-0.2 when eluted with conc. NH₄OH/MeOH (2/5) solution. The final product with Cl⁻ salt can be prepared with an ion-exchange column packed with Dowex 1X8-200 (Cl⁻ form) and eluting with water. After collected the desired fractions and removal of solvent, the final products are subjected to bioassay directly confirming the synthesis and recovery of 6-O-(6-Deoxy-4-O-n-octyl-α-D-glucopyranosyl)neamine (FG08).

### Example 2

### In Vitro MIC Test of the Antimicrobial Activity of 6-O-(6-Deoxy-4-O-n-octyl-α-D-glucopyranosyl)neamine (FG08)

The following results are representative of the antimicrobial activity of the compound of the present invention and are in no way meant to be limiting.

Referring now to Table 1, the MICs of 5 aminoglycosides, JL038 (FG08), JL39, JL40, kanamycin A, and kanamycin B, were estimated in sterile, flat-bottomed 96-well microtiter plates (Corning Costar, Corning, N.Y.) in the range of 500 to 1 μg/mL. Stock solutions were prepared at a concentration of 2 mg/mL in water. In a 96-well plate, 50 μL aliquots of each aminoglycoside stock solution was added in the column 3 wells and two-fold serial dilutions were made 10 times (to column 12) with sterile distilled water. Then, 40 μL of PDB+casamino acids and 10 μl of 10⁵ macroconidia/mL F. graminearum suspension were added to the wells. Negative (90 μl of PDB+casamino acids and 10 μL of sterile distilled water) and positive (90 of PDB+casamino acids and 10 μL of 10⁵ macroconidia/mL of F. graminearum) controls were placed in wells of columns 1 and 2, respectively. The plates were incubated at 24° C. for 72 h. The optical densities of the wells were measured using an ELx800 Absorbance Microplate Reader (BioTek Instruments Inc., Winooski, Va.) with a wavelength of 630 nm every 12 h after inoculation. The percentages of inhibition of fungal growth were calculated as follows: [(A630 of a well at 72 h)−(A630 of a well at 0 h)]/[(A630 of column 2 at 72 h)−(A630 of column 2 at 0 h)]×100%. MIC tests were replicated four times and each treatment was repeated at least two times.

Referring once again to Table 1, the concentrations of each aminoglycoside required to inhibit 90% of the growth or more (MIC₉₀) are summarized. JL39 and kanamycin A were not lethal to F. graminearum in the range of 500 to 1 μg/mL. JL40, and kanamycin B were fungicidal in the range of 250 to 125 μg/mL. The most active anti-Fusarium aminoglycoside tested was JL038 with an MIC of 31.3 μg/mL.

The structural difference between kanamycin A and kanamycin B is the occurrence of a hydroxyl or amino group at 2′ position of ring I, respectively. This difference may account for the difference in their antifungal activities. New aminoglycoside, JL038 and comparative aminoglycoside JL40, are kanamycin B derivatives which have different amino sugars in the ring III. Both have increased antifungal activity compared to kanamycin B (see Table 1). These two synthetic analogs represent successful improvement of antimicrobial activity of an aminoglycoside by glycodiversification.

JL038 (FG08), JL40, and kanamycin B were selected for further testing. The MICs of these three aminoglycosides were determined in PDB+casamino acids medium containing 0.02, 0.08 or 0.2% (v/v) surfactant. Surfactant did not affect the in vitro activities of all three aminoglycosides (see Table 1).

### Example 3

### In Planta Leaf Injection Assay of the Antimicrobial Activity of 6-O-(6-Deoxy-4-β-n-octyl-α-D-glucopyranosyl)neamine (FG08)

Referring now to FIG. 3, the in planta experiments use surfactant (C₅₈H₁₁₄O₂₆) as an adjuvant, and there are possibilities that this surfactant enhances, suppresses, or eliminates the activities of aminoglycosides. To test the effects of surfactant, MIC tests were done in the presence of surfactant. Aminoglycosides that showed activities against F. graminearum were tested with PDB+casamino acids and surfactant. Surfactant was mixed into PDB+casamino acids at concentrations of 0.05, 0.2, or 0.5% (v/v). Forty μL portions of these media were added to the microtiter plate wells to give 100 μL final volumes of F. graminearum-containing suspensions (10⁴ macroconidia/mL) under aminoglycoside treatments with final concentrations of surfactant of 0.02, 0.08, or 0.2% (v/v). The incubation and growth measurement procedures were the same as MIC tests without surfactant. JL038, JL40, and kanamycin B were tested for antifungal activity in planta. Because amounts were limited, the reagents were applied by mixing in inocula rather than spraying the entire plants. Solutions containing aminoglycosides at 30 μg/mL, 180 μg/mL, and 1080 μg/mL in 0.25% (w/v) agar and 0.2% (v/v) surfactant were prepared to test their phytotoxicities. The pathogen was grown on mung bean agar plates (9) for 7 days, and the suspension (2.0×10⁴ macroconidia/mL) was prepared in sterile 0.25% (w/v) agar solution with 0.2% (v/v) of surfactant. It was mixed 1:1 by volume with solutions containing either 60 μg/mL, 360 μg/mL or 2160 μg/mL of aminoglycoside in 0.25% (w/v) agar solution containing 0.2% (v/v) surfactant. Final inocula and aminoglycoside concentrations were 10⁴ macroconidia/mL and 30 μg/mL, 180 μg/mL or 1080 μg/mL, respectively. Non-treated, negative control plants, and positive control plants inoculated with 10⁴ macroconidia/mL in 0.25% (w/v) agar and 0.2% (v/v) surfactant solution, respectively, were also prepared. Eight replications were performed for each treatment for both Alsen and Frontana wheat cultivars. Each whole set of experiments was repeated three times.

After the measurements, the inoculated parts of the leaves were cut out as 5 mm length segments. The surfaces of the leaf segments were rinsed with 30% bleach and sterile distilled water two times, and placed on surfaces of PDA+casamino acid agar plates (15% w/v agar). The plates were incubated at 24° C. for 48 h and fungal growth from the segments determined by visual observation.

Referring again to FIG. 3, JL038, JL40, and kanamycin B were tested for their in planta antifungal activities against F. graminearum isolate B-4-5A. Each of JL038, JL40 and kanamycin B where tested at each of 30 μg/ml, 180 μg/mL and 1080 μg/mL. The y-axis of FIG. 3 show the percent lesion area observed, following a particular treatment. Each particular treatment is show on the x-axis. For example, on the x-axis of FIG. 3, “38-30” refers to treatment with JL038 at 30 μg/mL. The presence of the letter “F” at the end of the abbreviation indicates the treatment was carried out in the presence of. F. graminearum (for example, “38-30-F” indicates treatment with JL038 at 30 μg/mL1 in the presence of F. graminearum), whereas the absence of the letter “F” indicates the treatment was carried out in the absence of F. graminearum. This convention for abbreviation of treatment is followed for each treatment listed on the x-axis of FIG. 3. Furthermore, as per the legend associated with FIG. 3, the darker colored graph columns indicate a treatment performed on Frontana wheat cultivars, whereas lighter colored graph columns indicate a treatment performed on Alsen wheat cultivars.

Referring yet again to FIG. 3, although JL038, JL40, and kanamycin B all show activity in vitro, only JL038 shows in vivo fungicidal activities at concentrations that are also non-phytotoxic. For example, at 30 μg/mL JL038 prevents lesion development by F. graminearum while demonstrating no phytotoxicity. Leaf segments treated with higher levels of JL038 showed phytotoxicity, as indicated by chlorosis in the absence of fungal growth. Phytotoxicity may be cultivar dependent, as the Frontana cultivar was relatively resistant to JL038 phytotoxicity as compared to the Alsen cultivar.

Still referring to FIG. 3, there is shown the percent lesion areas developed in leaf infection assays with JL038, JL40 and kanamycin B treatments. The lesion areas were measured by digital photography and analysis with APS Assess (plant disease quantification software, APS Press, St Paul, Minn.). Aminoglycosides were applied to wheat seedlings at 30, 180, and 1080 μg/mL (in 2.5% [w/v] agar solution including 0.2% [v/v] surfactant) with and without F. graminearum macroconidia. Thirty μg/mL of JL038 prevented the lesion area developments while having no phytotoxicities.

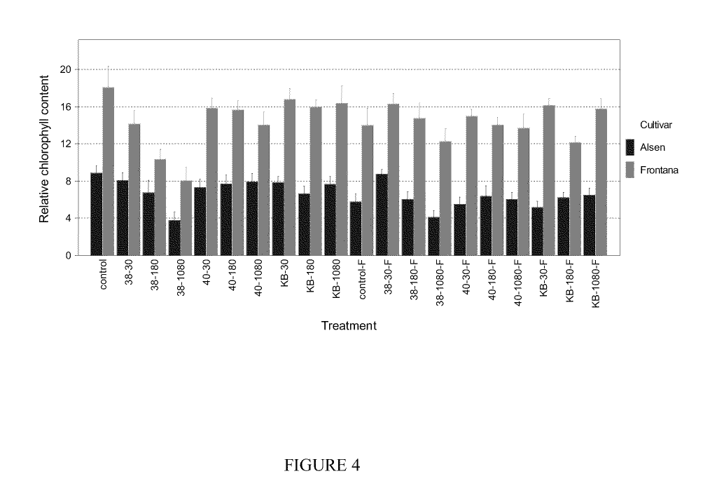

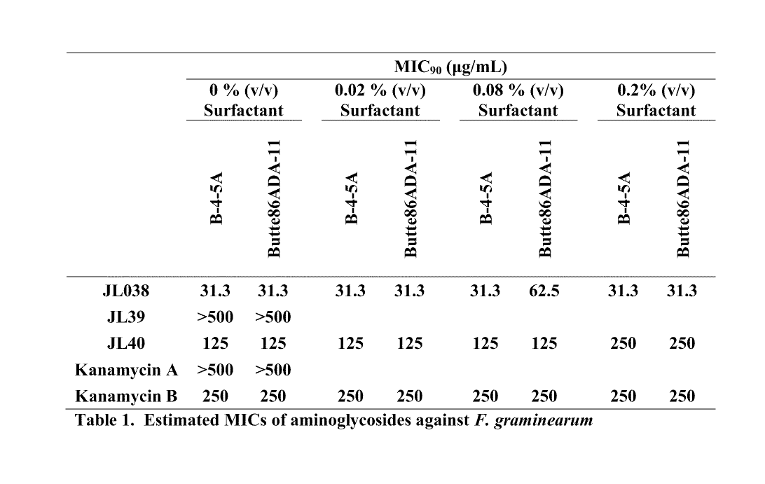

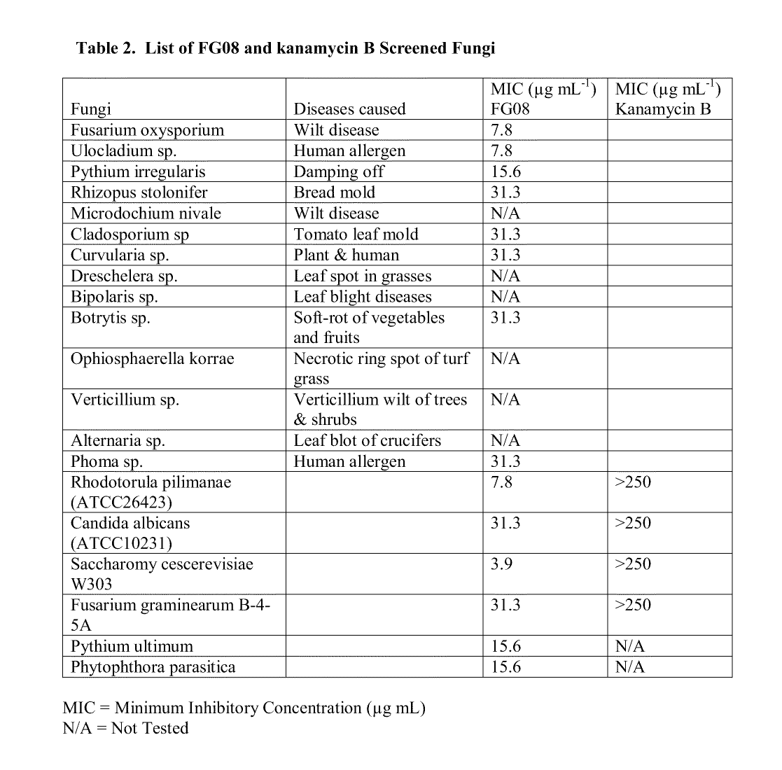

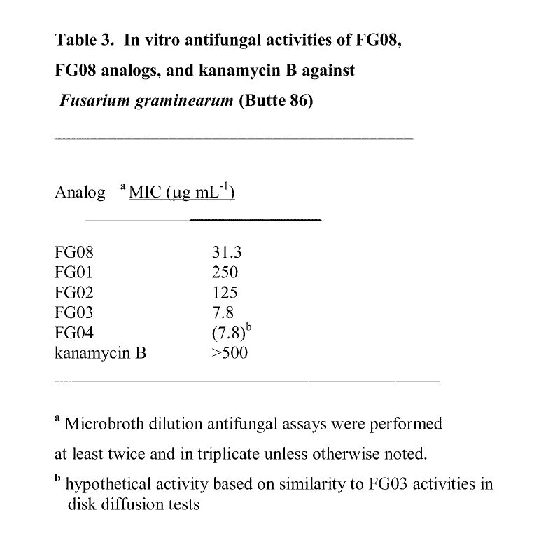

Referring now to FIG. 4, there are shown relative chlorophyll contents in leaf infection assays with JL038, JL40 and kanamycin B treatment. The y-axis of FIG. 4 shows the relative chlorophyll content following each particular treatment shown on the x-axis. The x-axis of FIG. 4 follows the same convention for abbreviation of treatment as discuss above for FIG. 3. Results of experiments measured using a CCM-200 Chlorophyll Content Meter (OPTICSCIENCE, Tyngsboro, Mass.) are shown. Aminoglycosides were applied to wheat seedlings at 30, 180, and 1080 μg/mL (in 2.5% [w/v] agar solution including 0.2% [v/v] surfactant) with and without F. graminearum. JL038 showed no chlorophyll damages at 30 μg/mL while it became phytotoxic at higher concentrations. JL40 and kanamycin B were phytotoxic at all tested concentrations.

In contrast to JL038, aminoglycosides JL40 and kanamycin B were phytotoxic at 30 μg/mL (see FIG. 3 and FIG. 4). Leaf segments treated with JL40 and kanamycin B at 30 μg/mL showed mycelial growth of F. graminearum, and macroconidia characteristic of Fusarium species were observed microscopically; therefore, at 30 μg/mL, JL40 and kanamycin B did not prevent leaf infection by F. graminearum.

In summary, JL038 controlled both in vitro and in vivo growth of F. graminearum, while JL40 and kanamycin B were effective only in vitro at higher concentrations. JL038 has been shown to have fungicidal but no antibacterial activities. It is structurally different from other kanamycin B analogs due to the presence of a carbon alkyl chain on ring III. The presence of a carbon alkyl chain on ring III might function in promoting the antifungal activity of JL038, which the parent molecule kanamycin B lacks. A fungal specific aminoglycoside drug such as JL038 will be beneficial in crop protection strategies because it would likely not promote bacterial resistance as do many conventional aminoglycosides.

### Example 4

### The Minimum Inhibitory Concentration (MIC) of JL038 Against Screened Fungi

The MIC for JL038 (FG08) was determined for various fungi and somewhat compared with kanamycin B. The results of the screening are listed in Table 2. JL038 was highly effective against all screened fungi. The lowest MICs (7.8 μg/mL) observed for JL038 were for treatment against Ulocladium sp. and Fusarium oxysporium. MICs for all species of screened fungi were at or below 31.3 μg/mL. The results listed in Table 2 demonstrate JL038 is a highly effective, broad spectrum antifungal agent whereas kanamycin B was substantially ineffective.

The advantages of the present invention include, without limitation, aminoglycoside analogs of kanamycin B and kanamycin A with improved fungicidal properties. The aminoglycoside analogs of the present invention demonstrates control of fungal pathogens both in vitro and in vivo. Specifically, the aminoglycoside analog FG08 (JL38) of the present invention controls the growth of F. graminearum both in vitro and in vivo, whereas both kanamycin B and M40 were able to control growth of F. graminearum only in vitro and only at higher concentrations than required for control of F. graminearum with FG08 (M38). The amimoglycoside analog of the present invention demonstrates no antibacterial activity and is structurally distinct from kanamycin B and most kanamycin B analogs due to the presence of a carbon alkyl chain on ring III. The carbon alkyl chain on ring III, absent on the parent molecule kanamycin B, is the structural feature of the present invention most likely responsible for the novel antifungal activity of the present invention. The fungal specificity of the present invention will benefit crop protection strategies because use of the present invention will not promote bacterial resistance, whereas conventional aminoglycosides do promote bacterial resistance.

While not as extensively tested, certain kanamycin A analogs, also substituted on ring III are expected to show similar properties.

### Example 5

### Selective Antimicrobial Activity of JL038 (FG08)

The minimum inhibitory concentration of M038 was determined for selected bacterial pathogens. For purposes of the experiments discussed in this paragraph, the minimum inhibitory concentration (MIC) is defined as the minimum concentration of compound needed to inhibit the growth of bacteria. A solution of selected bacteria was inoculated Trypticase Soy broth at 35° C. and incubated for 1-2 hours. Following incubation, the bacterial concentrations were determined, and diluted with broth, if necessary, to an absorption value of 0.08 to 0.1 at 625 nm. The adjusted inoculated medium (100 μL) was diluted with 10 ml broth, and then applied to a 96-well microtiter plate (50 μL). A series of solutions (50 μL each in 2-fold dilution) of M038 was added to the testing wells. The 96-well plate was incubated at 35° C. for 12-18 hrs. The MIC results were repeated at least three times. Minimal inhibitory concentration (MIC) values (μ/mL) for M038 (FG08) against the following bacteria are as follows: 125-250 for Escherichia coli TG1 (pET28a), 250 for Staphylococcus aureus ATCC 25923, 250 for Staphlococcus aureus (ATCC 33591), 250 for Pseudomonas aeruginosa (ATCC 27853), 125-250 for Enterococcus faecalis (ATCC29212), 250 for Klebsiella pneumoniae (ATCC 138883) and 250 for Klebsiella pneumonia (ATCC 700603). Corresponding MIC values for kanamycin B varied between <0.98 and 1.95 mg/ml. The MIC values determined for JL038 (FG08) exceed the values that typically prompt consideration of candidate compounds as effective antibacterial antibiotics (<16 μ/ml) whereas MIC values for kanamycin B demonstrated effective antibacterial activity.

### Example 6

### The Minimum Inhibitory Concentration (MIC) of FG08 and Analogs Against Fusarium graminearum as Compared to Kanamycin B

The MICs for FG08, FG01, FG02, FG03 and FG04 was determined for Fusarium graminearum (Butte 86) and compared to kanamycin B. The results of the screening are listed in Table 3. All showed antifungal activity varying from 7.8 to 250 μg/mL.

One preferred embodiment of the present invention is the treatment of fungal infection in a host in need thereof, where the elimination or reduction of bacteria associated with said host is undesirable. Without wishing to limit the scope of the invention in any way, one such use could occur in human or non-human mammals, where treatment of a fungal infection with and aminoglycoside of the invention such as FG08 (JL038) would eliminate or alleviate the fungal infection, but not affect the integrity of the intestinal flora of the host. Again, without limiting the invention, a second example is the treatment or prevention of fungal disease in a host crop, where it is undesirable to affect the diversity or abundance of bacteria of said host crop.

In broad embodiment, the present invention is drawn to novel antifungal compounds, a method to synthesize said novel antifungal compounds, and methods to use said novel antifungal compounds to treat humans, animals, soil, or plants to eliminate fungal growth and activity. In one broad embodiment, the structure related to the present invention is derived from a parent aminoglycoside molecule other than kanamycin B that is capable of being modified by the addition of a variety of substituents on ring III equivalent of the ring III of kanamycin B. Particularly preferred is the addition of a carbon alkyl chain as designated herein on ring III. In yet another broad embodiment the present invention is derived from the parent aminoglycoside molecule by the synthesis method shown herein, but the substituent, such as the carbon alkyl chain on ring III of the structure related to the present invention varies in the number of carbon atoms and hydrogen atoms. In still yet another broad embodiment, the present invention is used to treat a variety of fungal pathogens related to human, crop, or animal disease. In further broad embodiments, the compound of the present invention is administered by spraying, direct injection, topical application, ingestion (including pharmaceutical compositions the include the structure related to the present invention), or by inclusion in the water supply, to either a human, an animal, or a crop immediately threatened by, or potentially threatened by, a fungal pathogen, where fungal pathogen is causing or may cause fungal disease, and administration of the compounds of the present invention will reduce, eliminate, or avoid fungal disease.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed. Such embodiments may encompass different means of applying the compounds of the present invention, including, but not limited to, spraying, topical application, or injection. Various embodiments may also include the treatment different kinds of hosts susceptible to fungal infections. Types of hosts can include, but are not limited to, warm-blooded animals (including humans and other mammals), plants, fish, or bacterial cultures. Various embodiments may also include, but are not limited to, specific application methods. For instance, one example of a specific method of application to prevent or treat crop disease includes: producing a droplet of an antifungal compound between 250 and 400 microns, angling a spray nozzle forward 30 to 45 degrees down from horizontal, and applying said antifungal compound through said spray nozzle 5-14 inches above the grain head of a small grain host.

1. A fungicidal aminoglycoside compound, or a salt thereof, having the formula: 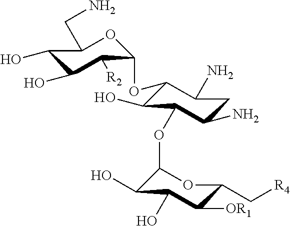
wherein R₄ is a member selected from the group consisting of H and OH; R₂ is a member selected from the group consisting of OH and NH₂; and R₁ is a member selected from the group consisting of R₃, R₃O(CO), R₃NH(CO), R₃S(O)₂, R₃S(O), R₃P)O₂R₃C(O)phenyl and C₁ to C₆ alkyl substituted phenyl; wherein R₃ is a straight or branched chain C₄ to C₁₂ alkyl group.

2. A compound according to claim 1 wherein R₁ is R₃.

3. A compound according to claim 2 wherein R₃ is straight chain C₄ to C₁₂ alkyl group.

4. A compound according to claim 3 wherein R₂ is NH₂.

5. A compound according to claim 4 wherein R₃ is n-octyl.

6. A compound according to claim 5 wherein R₄ is H.

7. A compound according to claim 5 wherein R₄ is OH.

8. A compound according to claim 3 wherein R₂ is OH.

9. A compound according to claim 8 wherein R₃ is n-octyl.

10. A compound according to claim 9 wherein R is OH.

11. A method of treating or preventing a fungal infection which comprises administering to a host in need thereof an effective amount of an aminoglycoside compound having the formula: 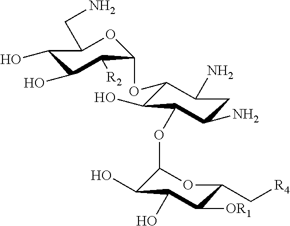
wherein R₄ is a member selected from the group consisting of H and OH; R₂ is a member selected from the group consisting of OH and NH₂; and R₁ is a member selected from the group consisting of R₃, R₃O(CO), R₃NH(CO), R₃S(O)₂, R₃S(O), R₃P)O)₂, R₃C(O), phenyl and C₁ to C₆ alkyl substituted phenyl; wherein R₃ is a straight or branched chain C₄ to C₁₂ alkyl group;
to a host in need thereof.

12. A method according to claim 11 wherein R₁ is R₃.

13. A method according to claim 12 wherein R₃ is straight chain C₄ to C₁₂ alkyl group.

14. A method according to claim 13 wherein R₂ is NH₂.

15. A method according to claim 14 wherein R₃ is n-octyl.

16. A method according to claim 15 wherein R₄ is H.

17. A method according to claim 16 wherein said host in need thereof is a plant.

18. A method according to claim 17 wherein said fungal infection is caused by F. graminearum.

19. A method according to claim 18 wherein said plant is a grain head.

20. A method according to claim 16 wherein said host in need thereof is a warm blooded animal.
